SEQUENCE LISTING (3 sequences)

SEQ ID NO: 1 - protein, 145 aa, Saccharomyces cerevisiae
MGKGKPRGLNSARKLRVHRRNNRWAENNYKKRLLGIAFKSSPFGGSSHAKGIVLEKLGIESKQPNSAIRKCVRVQLIKNGKKVTAFVPNDGCLNFVDENDEVLLAGFGRKGKAKGDIPGVRFKVVKVSGVSLLALWKEKKEKPRS

SEQ ID NO: 2 - protein, 143 aa, Cricetulus griseus [sequence omitted]

SEQ ID NO: 3 - protein, 143 aa, Homo sapiens [sequence omitted]

(19) United States
(12) Patent Application Publication
(10) Pub. No.: US 2018/0216153 A1
(43) Pub. Date: Aug. 2, 2018

(54) Biological Systems for Production of Highest Quality Proteins

(71) Applicant: Mediterranean Institute for Life Sciences, Split (HR)

(72) Inventors: Miroslav Radman, Hvar (HR); Anita Krisko, Varazdin (HR)

(21) Appl. No.: 15/702,198

(22) Filed: Sep. 12, 2017

Related U.S. Application Data
(63) Continuation of application No. 14/133,165, filed on Dec. 18, 2013, now abandoned.
(60) Provisional application No. 61/738,842, filed on Dec. 18, 2012.

(51) Int. Cl. C12P 21/00; C07K 14/195; C12N 9/26

(52) U.S. Cl. CPC C12P 21/00; C12N 9/2408; C07K 14/195

(57) ABSTRACT

Vaccines and therapeutic proteins, including polyclonal and monoclonal antibodies, must be maximally pure and stable in their most active native form. This is a requirement for their maximal efficacy, specificity and stability as well as for precluding immune responses against erroneous or damaged moieties. Similar considerations hold for proteins used in diagnostics, industry and research. The most frequent source of damage to proteins produced in living cells is the diverse product of oxidative damage. Two main sources of protein oxidation are the level of reactive oxygen species (ROS) and even more importantly the intrinsic susceptibility of proteins to oxidative damage. Methods for avoiding oxidative protein damage are disclosed, including providing for (i) a decrease in intracellular ROS levels and (ii) an increase in the intrinsic resilience of proteins to oxidative damage. Metabolites synthesized by the most robust species provide exceptionally high levels of protection against oxidative damage from ROS. High fidelity ribosomal mutations and over-expression of diverse chaperones increase the accuracy of protein biosynthesis and of protein post-synthetic folding, both greatly contributing to increased intrinsic resistance of proteins to oxidative damage.

### CROSS REFERENCES

This application claims the benefit of U.S. Provisional Application Ser. No. 61/738,842, filed Dec. 18, 2012, which is incorporated herein by reference in its entirety.

### FIELD OF THE INVENTION

The present invention in some embodiments relates to methods of improving protein quality by reducing the level of damage from intracellular oxidative species in a protein-producing cell line. The present invention in some embodiments also relates to reducing the level of damage from intracellular oxidative species by providing improved intrinsic protection to a protein. The present invention in some embodiments further relates to reducing the level of damage from intracellular oxidative species by providing methods of reducing intracellular oxidative species. Furthermore, the present invention in some embodiments relates to the following methods, alone or in combination, of reducing intracellular oxidative species or providing improved intrinsic protection from intracellular oxidative species to a protein: cloning a gene from a polyextremophile such as D. radiodurans into another species, overexpressing a chaperone, and introducing a high-fidelity ribosomal mutation. The present invention in some embodiments also relates to methods of producing proteins using a polyextremophile such as Deinococcus radiodurans (D. radiodurans), in which intracellular proteins are protected from oxidative damage more effectively than in standard species.

### BACKGROUND OF THE INVENTION

There is a growing need to produce the highest quality proteins for use in medicine, research, and industry. In particular, industrial enzymes and diverse therapeutic proteins, e.g. immunoglobulins, monoclonal antibodies, and vaccines, need to be maximally pure, active, specific and stable. The limits to the quality of proteins produced naturally or by genetic engineering in bacteria, cell cultures, or in living organisms are caused by biosynthetic and folding errors and, more importantly, by protein damage incurred in the host cells and during their biosynthesis and purification procedures. Chemical modifications induced by oxygen free radicals, such as reactive oxygen species (“ROS”) and reactive nitrogen species (“RNS”), are the predominant source of protein damage. The impact of the so far unavoidable oxidative damage to proteins is so great that a number of cellular systems have evolved to (i) diminish the production or lifetime of ROS, (ii) revert or repair the incurred damage and (iii) eliminate damaged proteins by selective proteolysis. The most reactive ROS is the hydroxyl radical (°OH) and the most deleterious irreversible protein damage is the carbonylation of amino acid side chains.

With the incurred intrinsic and environmentally-inflicted oxidative stress in the course of cellular life, the steady-state of non-reparable protein oxidation (carbonylation) and the selective breakdown of carbonylated proteins (by the dedicated proteases: bacterial Lon protease and mammalian 20S proteasome) leads to the accumulation of oxidized proteins as documented in all tested species. When these proteases become themselves inactivated by oxidative damage or by saturation, due to the excess of oxidized proteins, for example during stress caused by metabolic events or irradiation, cellular proteins undergo extensive oxidation. Non-degraded oxidized proteins tend to form aggregates—an adaptation that avoids the dominant negative interference with the activity of their undamaged counterparts. This basic biological chemistry of cellular proteins is particularly relevant to the over-expressed proteins destined for commercial use.

### SUMMARY OF THE INVENTION

In an embodiment, the inventors have identified a bacterial species—the polyextremophile Deinococcus radiodurans (D. radiodurans)—in which all intracellular proteins are protected from oxidative damage much more effectively than in standard species. That protection from oxidation is the key element of the robustness of D. radiodurans compared to other biological species.

In an embodiment, the inventors associate three specific elements that each separately and/or in various combinations with one another, and at different levels, improve the quality of synthesized proteins and in various embodiments, act in synergy: (1) D. radiodurans, with its uniquely effective protection system against protein oxidation, as host cells for production of proteins of interest; (2) Ribosomal rpsL mutation that decreases the natural error rates in protein biosynthesis; and (3) Plasmid-born (or chromosomal) chaperone over-expression locus, including but not limited to tig, dnaK and groES/EL.

In an embodiment, disclosed herein is a method of producing a protein comprising over-expressing the protein in a cell line derived from a polyextremophile or D. radiodurans.

In an embodiment, disclosed herein is a method of producing a protein comprising over-expressing the protein in a cell line derived from a polyextremophile or D. radiodurans, wherein the over-expressed protein is protected from oxidative damage.

In an embodiment, disclosed herein is a method of producing a protein comprising over-expressing the protein in a cell line derived from polyextremophile or D. radiodurans, wherein the over-expressed protein is protected from carbonylation.

In an embodiment, disclosed herein is a method of producing a protein comprising over-expressing the protein in a cell line derived from a polyextremophile or D. radiodurans, wherein the method further comprises the step of performing a post-synthetic modification to the over-expressed protein.

In an embodiment, disclosed herein is a method of preventing or reducing oxidative degradation in a protein-producing cell line comprising cloning a gene of a polyextremeophile or D. radiodurans so that the gene is expressed in the cell line.

In an embodiment, disclosed herein is a method of preventing or reducing oxidative degradation in a protein-producing cell line comprising cloning a gene of a polyextremophile or D. radiodurans so that the gene is expressed in the cell line, wherein the cell line is a Chinese hamster ovary cell line or an E. coli cell line.

In an embodiment, disclosed herein is a method of preventing or reducing oxidative degradation in a protein-producing cell line comprising cloning a gene of a polyextremophile or D. radiodurans so that the gene is expressed in the cell line, wherein the cell line is a prokaryotic bacterial cell line, a eukaryotic yeast cell line, or a eukaryotic mammalian cell line.

In an embodiment, disclosed herein is a method of preventing or reducing oxidative degradation in a protein-producing cell line comprising the step of introducing a high-fidelity ribosomal mutation or a ribosomal rpsL mutation.

In an embodiment, disclosed herein is a method of preventing or reducing oxidative degradation in a protein-producing cell line comprising the step of introducing over-expression of a chaperone or a plasmid-born chaperone over-expression locus.

In an embodiment, disclosed herein is a method of preventing or reducing oxidative degradation in a protein-producing cell line comprising any of the steps, alone or in combination, of introducing over-expression of a chaperone, introducing a high-fidelity ribosomal mutation, or cloning a gene of a polyextremeophile so that the gene is expressed in the cell line.

In an embodiment, disclosed herein is a method of preventing or reducing oxidative degradation in a protein-producing cell line comprising any of the steps, alone or in combination, of introducing over-expression of a chaperone, introducing a high-fidelity ribosomal mutation, or cloning a gene of a polyextremeophile so that the gene is expressed in the cell line, wherein the method further comprises the step of performing a post-synthetic modification to the over-expressed protein.

### BRIEF DESCRIPTION OF THE DRAWINGS

The foregoing summary, as well as the following detailed description of the invention, will be better understood when read in conjunction with the appended drawings.

FIG. 1 illustrates the results of Western blot analysis of the total protein carbonylation from non-irradiated and irradiated E. coli and D. radiodurans cells. The dose of delivered gamma radiation is shown at the bottom of the figure. The results depict the pattern of carbonylation of cellular proteins in D. radiodurans compared to bacterium E. coli.

FIG. 2A shows that cell death induced by ionizing gamma radiation correlates with protein carbonylation in E. coli (“Eco”) and D. radiodurans (“Dra”).

FIG. 2B shows that cell death induced by ultraviolet (“UV”) radiation correlates with protein carbonylation in E. coli (“Eco”) and D. radiodurans (“Dra”).

FIG. 2C shows that the ROS production induced by gamma radiation is delayed in D. radiodurans (“Dra”) to higher doses, even in the gamma-sensitive Dra mutant, ΔrecA, lacking one of the major post-irradiation DNA-repair proteins. This result suggests the presence of the ROS scavenger in Dra cells.

FIG. 2D shows that the ROS production induced by UV radiation is delayed in D. radiodurans (“Dra”) to higher doses, even in the UV-sensitive Dra mutant, ΔrecA, lacking one of the major post-irradiation DNA-repair proteins. This result suggests the presence of the ROS scavenger in Dra cells.

FIG. 3 illustrates the protein carbonylation increase observed with UV radiation 120 doses applied to bacterial cultures, with saturation observed below wild type (“wt”) values with decreasing levels of translation errors (rpsL mutation) and increasing chaperone (over-expression) activity in isogenic E. coli strains. The results represent the mean of 3 triplicate experiments with the standard deviation shown. The abbreviation “oe” stands for over-expression.

FIG. 4 shows that alpha-glucosidase purified from the E. coli rpsL141 exhibits an activity higher than the alpha-glucosidase produced in wild type E. coli MG1655 and is also more active than the alpha-glucosidases purchased from Megazyme and Sigma.

FIG. 5 shows that beta-glucanase from the E. coli rpsL141 displays an activity higher than the beta-glucanase produced in wild type E. coli MG1655 and the beta-glucanase purchased from Sigma. However, beta-glucanase purchased from Megazyme displays the highest activity under the tested conditions.

### DETAILED DESCRIPTION OF THE INVENTION

The present invention in some embodiments concerns methods of producing proteins using D. radiodurans as a host cell, in which the produced intracellular proteins are protected from oxidative damage more effectively than in standard species. The present invention in some embodiments also concerns methods of cloning genes from D. radiodurans into other host species, which can be used to produce proteins that are protected from oxidative damage normally encountered in the host species.

The nature of protection has not been identified with certainty but the exceptionally high manganese to iron (Mn++/Fe++) ratio in D. radiodurans could be contributing to the lower ° OH radical production than in other bacteria with orders of magnitude lower Mn++/Fe++ ratios (Daly M J, Gaidamakova E K, Matrosova V Y, Vasilenko A, Zhai M, Leapman R D, Lai B, Ravel B, Li S M, Kemner K M, Fredrickson J K. 2007. PLOS Biol. e92. Protein oxidation implicated as the primary determinant of bacterial radioresistance.). Irrespective of the mechanism of protein protection, D. radiodurans clearly produces proteins with 10 times higher quality than E. coli—the standard bacterial host for protein production.

This high protection against oxidative damage of the D. radiodurans proteome (as compared with E. coli) is not due to the intrinsic resistance of deinoccocal proteins but to the presence of small molecular weight molecules that neutralize ROS and protect equally well the “foreign” E. coli proteins. Cell-free extracts were prepared and irradiated either separately or in a 1:1 (E. coli+D. radiodurans) mixture (with or without previous dialysis by filtration), and the respective protein carbonylation was quantified as given in Table 1. E. coli protein carbonylation levels in extracts are similar to those obtained with irradiated cells. In the non-irradiated mixed (E. coli+D. radiodurans) extracts, high constitutive protein carbonylation levels of E. coli predominate, irrespective of dialysis. In irradiated mixed (E. coli+D. radiodurans) extracts, low levels of D. radiodurans protein carbonylation are dominant, showing the D. radiodurans protective effect on the E. coli proteome in vitro. This protection is lost with dialysis. Thus, proteins from other species over-expressed in D. radiodurans, even if susceptible to oxidation, will be protected against oxidative damage. Growth of D. radiodurans in the rich TGY medium yields less protein carbonylation than growth in the supplemented minimal medium. The active ingredient of proteome protection against oxidation is a cocktail of small molecular weight (<3 kDa) metabolites suppressing ROS activity and thereby protecting equally effectively proteins from diverse species (Krisko A, Radman M. 2010. Proc. Natl. Acad. Sci. USA 107: 14373-14377. Protein damage and death by radiation in Escherichia coli and Deinococcus radiodurans.).

TABLE 1
Protein carbonylation in γ- and UV-irradiated (UV C wavelength
range) E. coli and D. radiodurans cell extracts. The results
presented are the mean ± the standard deviation of four measurements.

| | Carbonyls/mg of protein (nmol) | | | |
|---|---|---|---|---|
| | Dose of UV | | Dose of gamma | |
| | radiation (J/m²) | | radiation (Gy) | |
| Cell extracts | 0 | 270 | 0 | 800 |

E. coli

| | | | | |
|---|---|---|---|---|
| non-dialyzed | 1.05 ± 0.12 | 7.25 ± 0.52 | 1.69 ± 0.22 | 5.14 ± 0.43 |
| dialyzed | 1.1 ± 0.21 | 7.84 ± 0.48 | 1.62 ± 0.24 | 5.17 ± 0.45 |

E. coli + D. radiodurans

| | | | | |
|---|---|---|---|---|
| non-dialyzed | 0.99 ± 0.17 | 1.95 ± 0.29 | 1.54 ± 0.17 | 1.92 ± 0.11 |
| dialyzed | 1.03 ± 0.14 | 9.15 ± 0.61 | 1.36 ± 0.11 | 6.12 ± 0.39 |

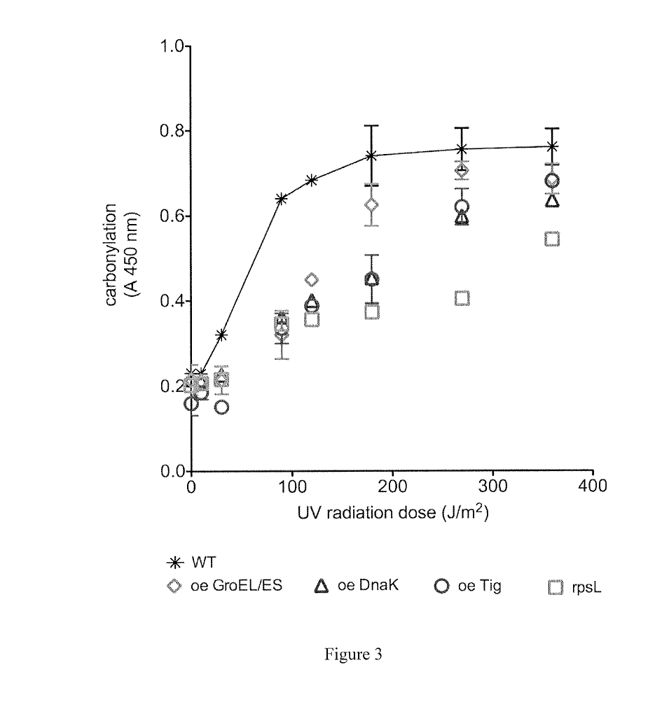

Protein structure has evolved, at a gene level, so as to minimize susceptibility to oxidative damage and thereby prolong the lifetime of proteins and their activity. Increasing protein biosynthetic error rates by drugs (e.g., streptomycin and puromycin) or mutations (rpsD) in ribosomal proteins, or decreasing the protein folding accuracy by deletion of chaperone proteins, leads to increased susceptibility of proteins to oxidative damage and carbonylation. The results in FIG. 3 show that protein carbonylation increases with the UV radiation dose applied to bacterial cultures and saturates at a level below that of the wild type values with decreasing levels of translation errors (rpsL mutation) and increasing chaperone over-expression) activity in isogenic E. coli strains. FIG. 3 shows that reciprocally, it is possible to improve the quality of the E. coli proteome by appropriate ribosomal high fidelity mutants (such as rpsL) or by chaperone over-expression, both leading—at constant ROS levels—to decreased levels of protein oxidative damage. Thus, the results illustrate the reduction of damaged proteins by about ten-fold by ROS reduction and at least as much by decreasing the susceptibility of proteins to oxidative damage.

The functional defects of proteins, correlating with their oxidative damage (carbonylation), can be suppressed by antioxidants in proportion to the extent of the decrease in protein carbonylation. This is illustrated by the effects of trolox (6-hydroxy-2,5,7,8-tetramethylchroman-2-carboxylic acid) on the reduction of ROS species levels and PC levels as given in Table 2.

TABLE 2
A summary of the effect of 1 mM trolox on reduction
of reactive oxygen species (ROS) level and
the protein carbonylation (PC) amount.

1 mM trolox effect

| Strain | ROS reduction | PC reduction |
|---|---|---|
| wt | 0.41 | 0.26 |
| Δtig | 0.45 | 0.33 |
| ΔdnaK | 0.41 | 0.25 |

In an embodiment, the inventors have identified a bacterial species—the polyextremophile Deinococcus radiodurans (D. radiodurans)—in which all intracellular proteins are protected from oxidative damage much more effectively than in standard species. That protection from oxidation is the key element of the robustness of D. radiodurans compared to other biological species.

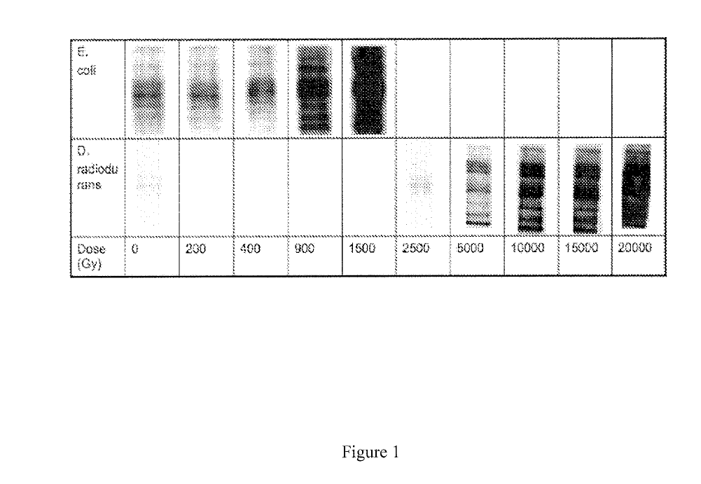

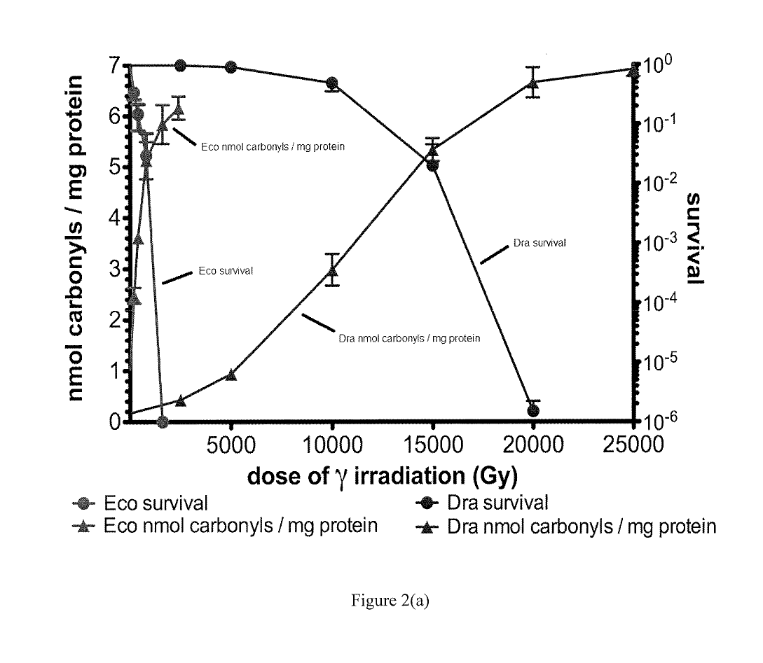

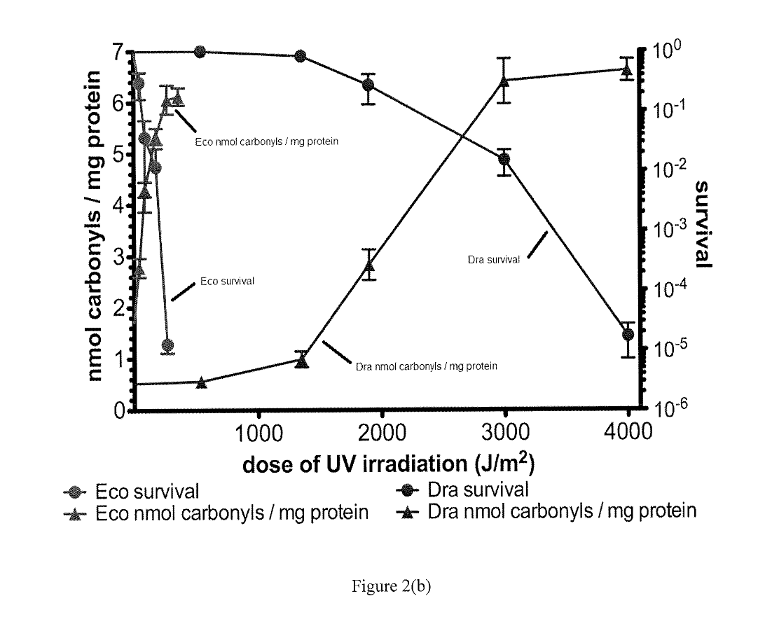

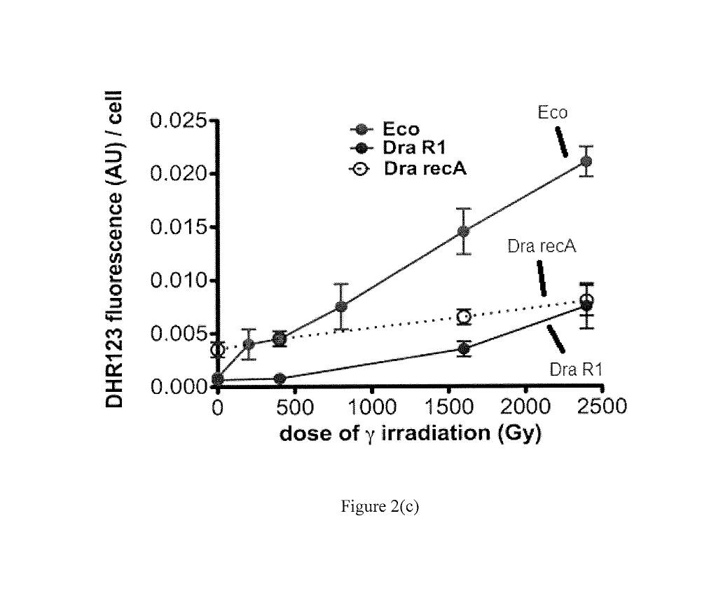

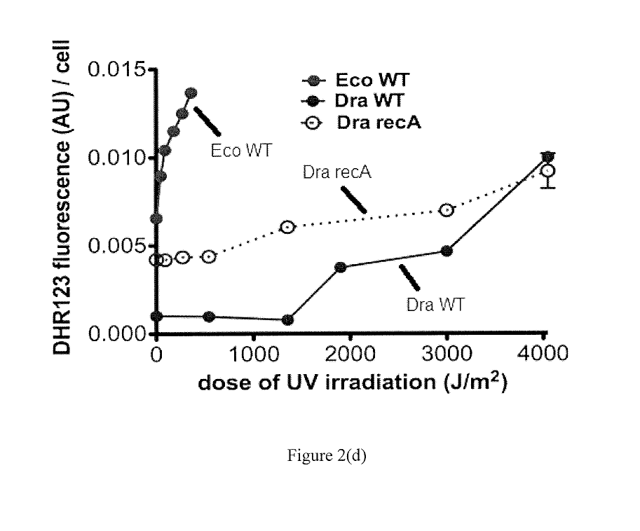

In various embodiments, the inventors associate three specific elements that each separately and/or in various combinations with one another, and at different levels, improve the quality of synthesized proteins and in various embodiments, act in synergy: (1) D. radiodurans, with its uniquely effective protection system against protein oxidation, as host cells for production of proteins of interest (FIGS. 1 and 2, Table 1); (2) Ribosomal rpsL mutation that decreases the natural error rates in protein biosynthesis (FIG. 3); and (3) Plasmid-born (or chromosomal) chaperone over-expression locus, including but not limited to tig, dnaK and groES/EL (FIG. 3).

In an embodiment, disclosed herein is a method of producing a protein comprising over-expressing the protein in a cell line derived from D. radiodurans.

In an embodiment, disclosed herein is a method of producing a protein comprising over-expressing the protein in a cell line derived from D. radiodurans, wherein the over-expressed protein is protected from oxidative damage.

In an embodiment, disclosed herein is a method of producing a protein comprising over-expressing the protein in a cell line derived from D. radiodurans, wherein the over-expressed protein is protected from carbonylation.

In an embodiment, disclosed herein is a method of producing a protein comprising over-expressing the protein in a cell line derived from D. radiodurans, wherein the method further comprises the step of performing a post-synthetic modification to the over-expressed protein.

In an embodiment, disclosed herein is a method of producing a protein comprising over-expressing the protein in a cell line derived from a polyextremophile.

In an embodiment, disclosed herein is a method of producing a protein comprising over-expressing the protein in a cell line derived from a polyextremophile, wherein the over-expressed protein is protected from oxidative damage.

In an embodiment, disclosed herein is a method of producing a protein comprising over-expressing the protein in a cell line derived from a polyextremophile, wherein the over-expressed protein is protected from carbonylation.

In an embodiment, disclosed herein is a method of producing a protein comprising over-expressing the protein in a cell line derived from a polyextremophile, wherein the method further comprises the step of performing a post-synthetic modification to the over-expressed protein.

In an embodiment, disclosed herein is a method of producing a protein in a cell line comprising the steps of (1) preventing or reducing the level of damage from at least one intracellular oxidative species in the cell line and (2) over-expressing the protein in the cell line.

In an embodiment, disclosed herein is a method of producing a protein in a cell line comprising the steps of (1) preventing or reducing the level of damage from at least one intracellular oxidative species in the cell line and (2) over-expressing the protein in the cell line, wherein the over-expressed protein produced by the method exhibits reduced damage.

In an embodiment, disclosed herein is a method of producing a protein in a cell line comprising the steps of (1) preventing or reducing the level of damage from at least one intracellular oxidative species in the cell line and (2) over-expressing the protein in the cell line, wherein the over-expressed protein produced by the method exhibits reduced protein carbonylation.

In an embodiment, disclosed herein is a method of preventing or reducing intracellular oxidative degradation in a protein-producing cell line comprising cloning a gene of a polyextremophile so that the gene is expressed in the protein-producing cell line.

In an embodiment, disclosed herein is a method of preventing intracellular oxidative degradation or protein carbonylation in a protein-producing cell line comprising cloning a gene of a polyextremophile so that the gene is expressed in the cell line, wherein the cell line is a prokaryotic bacterial cell line, a eukaryotic yeast cell line, or a eukaryotic mammalian cell line.

In an embodiment, disclosed herein is a method of preventing intracellular oxidative degradation or protein carbonylation in a protein-producing mammalian cell line comprising cloning a gene of a polyextremophile so that the gene is expressed in the mammalian cell line, wherein the mammalian cell line is a Chinese hamster ovary (“CHO”) cell line, a baby hamster kidney (“BHK”) cell line, a lymphoblastoid tumor cell line, a melanoma cell line, or a hybridized tumor cell line.

In an embodiment, disclosed herein is a method of preventing intracellular oxidative degradation or protein carbonylation in a protein-producing yeast cell line comprising cloning a gene of a polyextremophile so that the gene is expressed in the yeast cell line, wherein the yeast cell line is a Pichia pastoris cell line, a Saccharomyces cerevisiae cell line, or a Myceliophthora thermophila cell line.

In an embodiment, disclosed herein is a method of preventing intracellular oxidative degradation or protein carbonylation in a protein-producing bacterial cell line comprising cloning a gene of a polyextremophile so that the gene is expressed in the bacterial cell line, wherein the bacterial cell line is an E. coli cell line, Streptomyces lividans cell line, a Streptomyces griseus cell line, a Mycobacterium smegmatis cell line, a Corynebacterium glutamicum cell line, a Corynebacterium ammoniagenes cell line, a Brevibacterium lactofermentum cell line, a Bacillus subtilis cell line, a Bacillus megaterium cell line, a Bacillus licheniformis cell line, a Bacillus amyloliquefaciens cell line, a Lactococcus lactis cell line, a Lactobacillus plantarum cell line, a Lactobacillus casei cell line, a Lactobacillus reuteri cell line, or a Lactobacillus gasseri cell line.

In an embodiment, disclosed herein is a method of preventing intracellular oxidative degradation or protein carbonylation in a protein-producing cell line comprising cloning a gene of a polyextremophile so that the gene is expressed in the cell line, wherein the damage to the 275 protein produced by the cell line is diminished or the activity of the protein produced by the cell line is increased by at least about 1.1 times, at least about 1.2 times, at least about 1.3 times, at least about 1.4 times, at least about 1.5 times, at least about 1.6 times, at least about 1.7 times, at least about 1.8 times, at least about 1.9 times, at least about 2 times, at least about 3 times, at least about 4 times, at least about 5 times, at least about 6 times, at least about 7 times, at least 280 about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In an embodiment, disclosed herein is a method of preventing or reducing intracellular oxidative degradation in a protein-producing cell line comprising cloning a gene of D. radiodurans so that the gene is expressed in the protein-producing cell line.

In an embodiment, disclosed herein is a method of preventing intracellular oxidative degradation or protein carbonylation in a protein-producing cell line comprising cloning a gene of D. radiodurans so that the gene is expressed in the cell line, wherein the cell line is a prokaryotic bacterial cell line, a eukaryotic yeast cell line, or a eukaryotic mammalian cell line.

In an embodiment, disclosed herein is a method of preventing intracellular oxidative degradation or protein carbonylation in a protein-producing mammalian cell line comprising cloning a gene of D. radiodurans so that the gene is expressed in the mammalian cell line, wherein the mammalian cell line is a CHO cell line, a BHK cell line, a lymphoblastoid tumor cell line, a melanoma cell line, or a hybridized tumor cell line.

In an embodiment, disclosed herein is a method of preventing intracellular oxidative degradation or protein carbonylation in a protein-producing yeast cell line comprising cloning a gene of D. radiodurans so that the gene is expressed in the yeast cell line, wherein the yeast cell line is a Pichia pastoris cell line, a Saccharomyces cerevisiae cell line, or a Myceliophthora thermophila cell line.

In an embodiment, disclosed herein is a method of preventing intracellular oxidative degradation or protein carbonylation in a protein-producing bacterial cell line comprising cloning a gene of D. radiodurans so that the gene is expressed in the bacterial cell line, wherein the bacterial cell line is an E. coli cell line, Streptomyces lividans cell line, a Streptomyces griseus cell line, a Mycobacterium smegmatis cell line, a Corynebacterium glutamicum cell line, a Corynebacterium ammoniagenes cell line, a Brevibacterium lactofermentum cell line, a Bacillus subtilis cell line, a Bacillus megaterium cell line, a Bacillus licheniformis cell line, a Bacillus amyloliquefaciens cell line, a Lactococcus lactis cell line, a Lactobacillus plantarum cell line, a Lactobacillus casei cell line, a Lactobacillus reuteri cell line, or a Lactobacillus gasseri cell line.

In an embodiment, disclosed herein is a method of preventing intracellular oxidative degradation or protein carbonylation in a protein-producing cell line comprising cloning a gene of D. radiodurans so that the gene is expressed in the cell line, wherein the damage to the protein produced by the cell line is diminished or the activity is increased by at least about 1.1 times, at least about 1.2 times, at least about 1.3 times, at least about 1.4 times, at least about 1.5 times, at least about 1.6 times, at least about 1.7 times, at least about 1.8 times, at least about 1.9 times, at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In an embodiment, disclosed herein is a method of preventing intracellular oxidative degradation or protein carbonylation in a protein-producing cell line comprising the step of introducing a high fidelity ribosomal mutation.

In an embodiment, disclosed herein is a method of preventing intracellular oxidative degradation or protein carbonylation in a protein-producing cell line comprising the step of introducing a high fidelity ribosomal mutation, wherein the high fidelity ribosomal mutation is a ribosomal rpsL mutation.

In an embodiment, disclosed herein is a method of preventing intracellular oxidative degradation or protein carbonylation in a protein-producing cell line comprising the step of introducing a high fidelity ribosomal mutation, wherein the cell line is a prokaryotic bacterial cell line, a eukaryotic yeast cell line, or a eukaryotic mammalian cell line.

In an embodiment, disclosed herein is a method of preventing intracellular oxidative degradation or protein carbonylation in a protein-producing mammalian cell line comprising the step of introducing a high fidelity ribosomal mutation, wherein the mammalian cell line is a CHO cell line, a BHK cell line, a lymphoblastoid tumor cell line, a melanoma cell line, or a hybridized tumor cell line.

In an embodiment, disclosed herein is a method of preventing intracellular oxidative degradation or protein carbonylation in a protein-producing yeast cell line comprising the step of introducing a high fidelity ribosomal mutation, wherein the yeast cell line is a Pichia pastoris cell line, a Saccharomyces cerevisiae cell line, or a Myceliophthora thermophila cell line.

In an embodiment, disclosed herein is a method of preventing intracellular oxidative degradation or protein carbonylation in a protein-producing bacterial cell line comprising the step of introducing a high fidelity ribosomal mutation, wherein the bacterial cell line is an E. coli cell line, Streptomyces lividans cell line, a Streptomyces griseus cell line, a Mycobacterium smegmatis cell line, a Corynebacterium glutamicum cell line, a Corynebacterium ammoniagenes cell line, a Brevibacterium lactofermentum cell line, a Bacillus subtilis cell line, a Bacillus megaterium cell line, a Bacillus licheniformis cell line, a Bacillus amyloliquefaciens cell line, a Lactococcus lactis cell line, a Lactobacillus plantarum cell line, a Lactobacillus casei cell line, a Lactobacillus reuteri cell line, or a Lactobacillus gasseri cell line.

In an embodiment, disclosed herein is a method of preventing intracellular oxidative degradation or protein carbonylation in a protein-producing cell line comprising the step of introducing a high fidelity ribosomal mutation, wherein the damage to the protein produced by the cell line is diminished or the activity of the protein produced by the cell line is increased by at least about 1.1 times, at least about 1.2 times, at least about 1.3 times, at least about 1.4 times, at least about 1.5 times, at least about 1.6 times, at least about 1.7 times, at least about 1.8 times, at least about 1.9 times, at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In an embodiment, disclosed herein is a method of preventing intracellular oxidative degradation or protein carbonylation in a protein-producing cell line comprising the step of over-expressing a chaperone.

In an embodiment, disclosed herein is a method of preventing intracellular oxidative degradation or protein carbonylation in a protein-producing cell line comprising the step of over-expressing a chaperone, wherein the step of over-expressing a chaperone comprises introducing a plasmid-born chaperone over-expression locus.

In an embodiment, disclosed herein is a method of preventing intracellular oxidative degradation or protein carbonylation in a protein-producing cell line comprising the step of over-expressing a chaperone, wherein the step of over-expressing a chaperone comprises introducing a plasmid-born chaperone over-expression locus selected from the group consisting of tig, dnaK and groES/EL.

In an embodiment, disclosed herein is a method of preventing intracellular oxidative degradation or protein carbonylation in a protein-producing cell line comprising the step of over-expressing a chaperone, wherein the cell line is a prokaryotic bacterial cell line, a eukaryotic yeast cell line, or a eukaryotic mammalian cell line.

In an embodiment, disclosed herein is a method of preventing intracellular oxidative degradation or protein carbonylation in a protein-producing mammalian cell line comprising the step of over-expressing a chaperone, wherein the mammalian cell line is a CHO cell line, a BHK cell line, a lymphoblastoid tumor cell line, a melanoma cell line, or a hybridized tumor cell line.

In an embodiment, disclosed herein is a method of preventing intracellular oxidative degradation or protein carbonylation in a protein-producing yeast cell line comprising the step of over-expressing a chaperone, wherein the yeast cell line is a Pichia pastoris cell line, a Saccharomyces cerevisiae cell line, or a Myceliophthora thermophila cell line.

In an embodiment, disclosed herein is a method of preventing intracellular oxidative degradation or protein carbonylation in a protein-producing bacterial cell line comprising the step of over-expressing a chaperone, wherein the bacterial cell line is an E. coli cell line, Streptomyces lividans cell line, a Streptomyces griseus cell line, a Mycobacterium smegmatis cell line, a Corynebacterium glutamicum cell line, a Corynebacterium ammoniagenes cell line, a Brevibacterium lactofermentum cell line, a Bacillus subtilis cell line, a Bacillus megaterium cell line, a Bacillus licheniformis cell line, a Bacillus amyloliquefaciens cell line, a Lactococcus lactis cell line, a Lactobacillus plantarum cell line, a Lactobacillus casei cell line, a Lactobacillus reuteri cell line, or a Lactobacillus gasseri cell line.

In an embodiment, disclosed herein is a method of preventing intracellular oxidative degradation or protein carbonylation in a protein-producing cell line comprising the step of over-expressing a chaperone, wherein the damage to the protein produced by the cell line is diminished or the activity of the protein produced by the cell line is increased by at least about at least about 1.1 times, at least about 1.2 times, at least about 1.3 times, at least about 1.4 times, at least about 1.5 times, at least about 1.6 times, at least about 1.7 times, at least about 1.8 times, at least about 1.9 times, 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 16 times, at least about 17 times, at least about 18 times, at least about 19 times, or at least about 20 times.

In an embodiment, disclosed herein is a method of preventing intracellular oxidative degradation or protein carbonylation in a protein-producing cell line comprising cloning a gene of D. radiodurans so that the gene is expressed in the cell line, wherein the cell line is a prokaryotic bacterial cell line, a eukaryotic yeast cell line, or a eukaryotic mammalian cell line that further comprises the step of introducing a plasmid-born chaperone over-expression locus.

In an embodiment, disclosed herein is a method of preventing intracellular oxidative degradation or protein carbonylation in a protein-producing cell line comprising cloning a gene of D. radiodurans so that the gene is expressed in the cell line, wherein the cell line is a prokaryotic bacterial cell line, a eukaryotic yeast cell line, or a eukaryotic mammalian cell line that further comprises the step of performing a post-synthetic modification to the over-expressed protein.

In various embodiments, encompassed herein are one or more genetic modifications that provide the required post-synthetic modification of therapeutic, diagnostic or industrial proteins (when such modification is required for their activity, specificity, stability or localization) in D. radiodurans or any cell system devoid of such required protein modification.

In various embodiments, encompassed herein are one or more genetic modifications that provide the required human therapeutic proteins in D. radiodurans, where the human genes encoding the required post-synthetic modification are introduced into D. radiodurans.

In various embodiments, disclosed herein are high quality proteins that are produced using the procedures described or encompassed herein.

The invention is further described by the following non-limiting examples.

### EXAMPLES

### Example 1

The procedure for the quantification of protein carbonylation by Western blot (FIG. 1) is as follows:

(1) Aliquots of bacterial strain samples are pelleted at 6000 g for 10 minutes. Supernatant is removed and the cells are re-suspended in 10 mM phosphate-buffered saline (“PBS”) supplemented with protease inhibitors. Cells are broken with 3 freeze-thaw cycles and the mechanical homogenizer. These steps are followed by a 20 minute centrifugation at 12000 g in order to remove cell debris.

(2) Lipids are removed from the sample by using lipid removal agent (“LRA”, Sigma 13360-U), 10 mg/100 uL of the sample for 1 h at room temperature. LRA is removed by 15 minute centrifugation at 10000 g.

(3) Derivatization of protein carbonyls is performed in solution. The same amount of proteins (15-20 μg) is taken from all samples. It should be noted that since the protein concentration is different in different samples, different volumes are usually taken from the samples. This is very important since next steps of derivatization depend on this volume. For example: if 5 μL of sample are taken, then 5 μL of 10% sodium dodecyl sulfate (“SDS”) should be added, followed by 10 μL of derivatization solution (2.4 mg/mL of 2,4-dinitrophenylhydrazine (“DNPH”) in trifluoracetic acid) and 15 minutes later 7.5 μL of neutralization solution (2M Tris buffer, pH 7.4). If the initial volume of the sample is different, then all other volumes to follow are resealed. Derivatized sample is stable for a week at 4° C. After all samples are derivatized, protein concentration is set to be the same in all samples. The protein concentration may be determined again.

(4) The derivatized sample is prepared for SDS-PAGE electrophoresis by adding β-mercaptoethanol and Laemmli buffer containing SDS and glycerol.

(5) Aliquots of this sample are loaded at the same time on two gels. Both gels are run at currents of 15 rnA per gel while the sample is in the stacking gel and currents of 25 rnA per gel when the sample enters the resolving gel.

(6) When steps (4) and (5) are done, one gel proceeds with silver staining and the other with Western blot.

(7) During the Western blot process, the proteins are transferred to a polyvinylidene (“PVDF”) membrane in a wet transfer system at 200 mA. The transfer lasts for 1 hour. Times longer than one hour were tested, but were not observed to have an effect on the amount of transferred proteins.

(8) The membrane is blocked over night in milk at 4° C.

(9) The membrane is soaked in primary antibody for 1 hour.

(10) The membrane is washed with 10 mM PBS supplemented with 0.1% Tween surfactant 3 times per 10 minutes.

(11) The membrane is soaked in secondary antibody.

(12) The membrane is washed again 3 times per 10 minutes in 10 mM PBS supplemented with 0.1% Tween. Finally, the membrane is washed for 15 minutes in PBS at pH 7.4.

(13) The membrane is ready to be developed, usually by autoradiography.

(14) The bands reveled upon such a procedure are compared to the bends reveled by silver staining.

(15) The membrane can be stored at −20° C.

### Example 2

The procedure for the quantification of protein carbonylation by ELISA method is as follows. The steps of protein extraction are as described in Example 1 (steps 1 and 2). Protein concentration is determined and adjusted so that the proteins are loaded into ELISA wells at 10 μg/mL. Adsorbed proteins are derivatized by using 12 μg/mL DNPH. Derivatization of adsorbed proteins is followed by detection of derivatized dinitrophenol (DNP)-carbonyl by a rabbit anti-DNP primary antibody (Sigma, D9656) and goat anti-rabbit secondary antibody conjugated to HRP (Jackson ImmunoResearch, 111-035-14). Stocks of antibodies were prepared at ˜1 ug/uL and used at 1:7000 dilutions. Subsequent incubation with enzyme substrate 3,3′,5,5′-tetramethylbenzidine resulted in a colored product that was quantified using a microplate reader with maximum absorbance at 450 nm.

### Example 3

By way of a non-limiting example, the synthetic pathway for the synthesis of the deinococcal pigment deinoxanthin can be introduced into any cell line. The pathway consists of several genes from D. radiodurans: GGPP synthase (DR1395), phytoene synthase (DR0862), phytoene desaturase (DR0861), lycopene cyclase (DR0801), C-1′,2′ hydratase (DR0091), C-3′,4′ desaturase (DR2250), carotene ketolase (DR0093), glucosytransferase (DR0089) and acyltransferase (DR0090) (Ting B, Hua Y. 2010, Trends in Microbiology 18: 512-520, Carotenoid biosynthesis in extremophilic Deinococcus-Thermus bacteria.). DRC0027 gene encoding for pyrroloquinoline-quinone synthase can also be introduced to enable the synthesis of pyrroloquinoline-quinone, a potent cytosolic scavenger (Misra H S, Khairnar N P, Bank A, Indira Priyadarsini K, Mohan H, Apte S K. 2004. FEBS Letters 578: 26-30. Pyrroloquinoline-quinone: a reactive oxygen species scavenger in bacteria.).

Furthermore, point mutations in the ribosomal protein S28, known to cause high fidelity translation in Saccharomyces cerevisiae, can be introduced into any mammalian cell line because of the highly conserved S28 protein. Table 3 shows the multiple sequence alignment between the yeast, hamster and human S28. Additionally, cellular chaperones, generalized and specific organellar, can be added on a stable expression system into the used cell line.

TABLE 3
Multiple sequence alignment between yeast (Socerevisiae), hamster (Cricetulus
griseus) and human (Homo sapiens) S28 protein of the 40S ribosomal subunit. The
lysine residue (K, underlined and labeled in bold font) shows the site of amino
acid change leading to improved fidelity of protein biosynthesis (K62T, K62N and
K62Q).

| | |
|---|---|
| gi\|259146618\|YEAST | MGKGKPRGLNSARKLRVHRRNNRWAENNYKKRLLGIAFKSSPFGGSSHAK |
| gi\|354483181\|HAMSTER | MG--KCRGLRTARKLRSHRRDQKWHDKQYKKAHLGTALKANPFGGASHAK |
| gi\|45067011\|HUMAN | MG--KCRGLRTARKLRSHRRDQKWHDKQYKKAHLGTALKANPFGGASHAK |
| | ** * ***.:***** ***:::* :::*** ** *:*:.****:**** |
| gi\|259146618\|YEAST | GIVLEKLGIESKQPNSAIRKCVRVQLIKNGKKVTAFVPNDGCLNEVDEND |
| gi\|354483181\|HAMSTER | GIVLEKVGVEAKQPNSAIRKCVRVQLIKNGKKITAFVPNDGCLNFIEEND |
| gi\|4506701\|HUMAN | GIVLEKVGVEAKQPNSAIRKCVRVQLIKNGKKITAFVPNDGCLNFIEEND |
| | ******:*:*:*********************:************::*** |
| gi\|259146618\|YEAST | EVLLAGFGRKGKAKGDIPGVREKVVKVSGVSLLALWKEKKEKPRS |
| gi\|354483181\|HAMSTER | EVLVAGFGRKGHAVGDIPGVRFKVVKVANVSLLALYKGKKERPRS |
| gi\|4506701\|HUMAN | EVLVAGFGRKGHAVGDIPGVRFKVVKVANVSLLALYKGKKERPRS |
| | ***:*******:* *************:.******:* ***:*** |

### Example 4

By way of a non-limiting example, the process for expression of exo-alpha-1,4-glucosidase from Bacillus stearothermophilus and beta-D-glucanase from Aspergillus niger in E. coli expression systems is described. The genes encoding for exo-alpha-1,4-glucosidase from Bacillus stearothermophilus and beta-D-glucanase from Aspergillus niger were synthesized and 510 cloned into apD441 expression vector with a 1×FLAG peptide at the 3′ end (purchased from DNA 2.0). The constructs were transformed into E. coli MG1655 and E. coli rpsL141. Overnight cultures were diluted 200 times and grown until OD (600 nm) of 0.2. Then, isopropyl β-D-1-thiogalactopyranoside (IPTG) was added (0.5 mM final) in order to induce the expression of the proteins from the pD441 vector, followed by growing the culture to saturation. The cells were 515 harvested and washed three times in 10 mM PBS at pH 7.4.

Cells were lysed by 1 mg/mL lysozyme in 150 mM TRIS, pH 7.4, at 37° C. for 30 minutes. After the lysis step, cell debris was removed by a 20-minute centrifugation at 10,000×g. The remaining supernatant is mixed with Anti-FLAG Agarose Affinity Gel (Sigma) in the following manner:

(1) 40 uL of the gel suspension is washed 3 times in the 150 mM TRIS pH 7.4 by centrifugation at 1,500×g for 30 s.

(2) The washed resin is mixed with the protein extract and incubated over-night at 4° C. with shaking.

(3) This is followed by 3 cycles of washing with 150 mM TRIS pH 7.4 and an elution step.

(4) Elution is performed by exposing the resin (which now carries the FLAG-tagged protein) to 3M MgCl₂ during 1 hour at 4° C. The resin is pelleted at 1,500×g during 30 s.

(5) The supernatant is collected and the protein concentration is measured by using the BCA method (Pierce). Purity of the FLAG-tagged protein is checked by SDS-PAGE protein electrophoresis.

(6) The FLAG-tagged protein is transferred into 10 mM PBS pH 7.4 on Centricon Centrifugal Filter Units (Millipore) with a 3 kDa cutoff before the activity is measured.

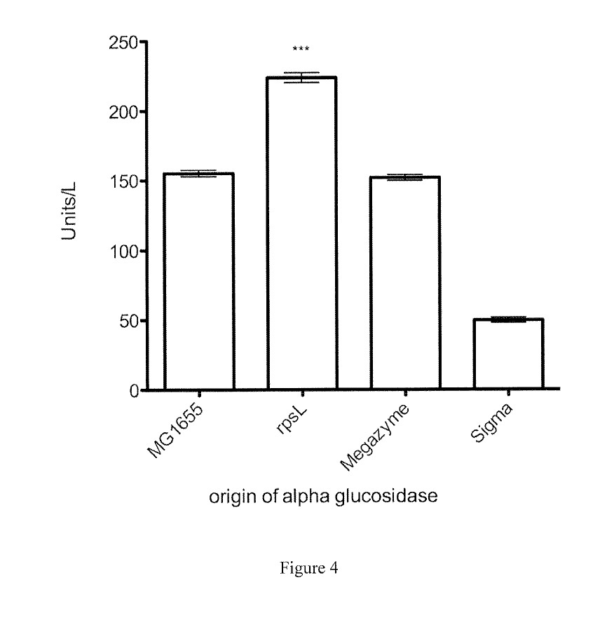

The activity of exo-alpha-1,4-glucosidase enzymes from Bacillus stearothermophilus produced in the two E. coli systems described above was compared to the activity of exo-alpha-1,4-glucosidase purchased from Megazyme (E-TSAGS) and Sigma (G3651). All proteins were transferred into 10 mM PBAS pH 7.4 by using Centricon Centrifugal Units with a 3 kDa cutoff. The concentration of each enzyme was adjusted to 0.5 mg/mL and the activity was measured by using the Abnova Alpha-glucosidase Assay kit (KA1608). Briefly, alpha-glucosidase hydrolyzes the terminal, non-reducing 1,4-linked alpha-D-glucose residues with release of alpha-D-glucose. The improved method utilizes p-nitrophenyl-α-D-glucopyranoside that is hydrolyzed specifically by alpha-glucosidase into a yellow colored product (maximal absorbance at 405 nm). The rate of the reaction is directly proportional to the enzyme activity. The results are summarized in FIG. 4, where alpha-glucosidase purified from the E. coli rpsL141 displays an activity higher than the one produced in wild type E. coli MG1655 and is also more active than the enzymes purchased from Megazyme and Sigma.

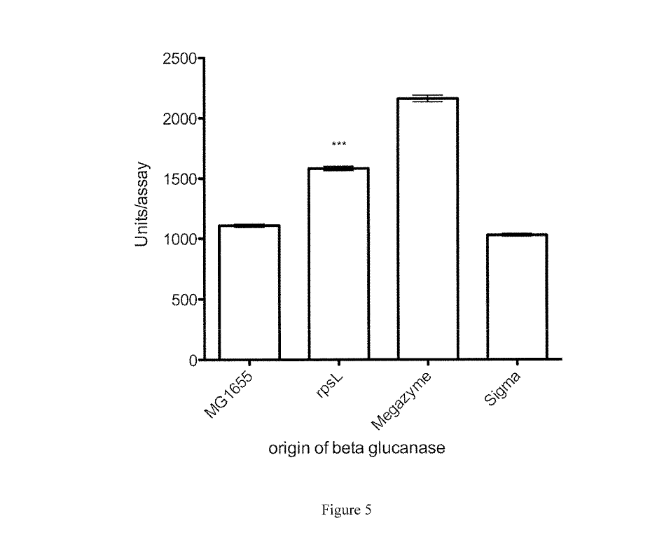

The activity of beta-D-glucanase enzymes from Aspergillus niger produced in the two E. coli systems described above was compared to the activity of beta glucanase purchased from Megazyme (E-CELAN) and Sigma (49101). All proteins were transferred into 10 mM PBAS with pH 7.4 by using Centricon Centrifugal Units with a 3 kDa cutoff. The concentration of each enzyme was adjusted to 0.5 mg/mL and the activity was measured by using the Azo-Barley Glucan Method (Megazyme, S-ABG100). Each protein is incubated with Azo-Barley glucan substrate under defined conditions. The dyed substrate is depolymerised by malt beta-glucanase to fragments which are soluble in the presence of precipitant solution. On centrifugation of the precipitant-treated reaction mixture, the absorbance (at 590 nm) of the supernatant solution is directly related to the activity of beta-glucanase in the sample. The results are presented in FIG. 5, where beta-glucanase from the E. coli rpsL141 displays an activity higher than that produced in wild type E. coli MG1655 and the enzyme purchased from Sigma. However, beta-glucanase from Megazyme displays the highest activity under the tested conditions.

In all cases, the statistical significance of the obtained results has been determined by using two-tailed t-tests.

What is claimed is:

1. A method of producing a protein in a cell line comprising the steps of: (1) reducing the level of damage from at least one intracellular oxidative species and (2) over-expressing the protein in the cell line.

2. The method of claim 1, wherein the step of reducing the level of damage from at least one intracellular oxidative species comprises the step of cloning a gene of D. radiodurans so that the gene is expressed in the cell line.

3. The method of claim 2, wherein the method further comprises the step of introducing a ribosomal rpsL mutation.

4. The method of claim 3, wherein the method further comprises the step of over-expressing a chaperone.

5. The method of claim 4, wherein the chaperone is selected from the group consisting of tig, dnaK, or groES/EL.

6. The method of claim 1, wherein the step of reducing the level of damage from at least one intracellular oxidative species comprises the step of introducing a ribosomal rpsL mutation.

7. The method of claim 6, wherein the method further comprises the step of over-expressing a chaperone.

8. The method of claim 7, wherein the chaperone is selected from the group consisting of tig, dnaK, or groES/EL.

9. The method of claim 1, wherein the step of reducing the level of damage from at least one intracellular oxidative species comprises the step of over-expressing a chaperone.

10. The method of claim 9, wherein the method further comprises the step of cloning a gene of D. radiodurans so that the gene is expressed in the cell line.

11. The method of claim 9, wherein the chaperone is selected from the group consisting of tig, dnaK, or groES/EL.

12. A method of producing a protein comprising over-expressing the protein in a cell line derived from D. radiodurans.

13. The method of claim 12, wherein the over-expressed protein is protected from oxidative damage.

14. The method of claim 12, wherein the over-expressed protein is protected from carbonylation.

15. The method of claim 12, wherein the method further comprises the step of performing a post-synthetic modification to the over-expressed protein.
